SEQUENCE LISTING (5 sequences)

SEQ ID NO: 1 - protein, 96 aa, Homo sapiens
  IGLV3-21 Allel 01
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDH

SEQ ID NO: 2 - DNA, 29 bp, Homo sapiens
tgtgctggtcatctattatgatagcgacc

SEQ ID NO: 3 - DNA, 29 bp, Homo sapiens
tgtgctggtcgtctatgatgatagcgacc

SEQ ID NO: 4 - DNA, 18 bp, Homo sapiens
agacggccaggattacct

SEQ ID NO: 5 - DNA, 20 bp, Homo sapiens
agagttggagccagagaatc

(19) United States
(12) Patent Application Publication
(10) Pub. No.: US 2022/0340978 A1
(43) Pub. Date: Oct. 27, 2022

(54) METHOD FOR DETECTING THE TENDENCY TO FORM LEUKEMIA

(71) Applicant: AVA Lifescience GmbH, Denzlingen (DE)

(72) Inventor: Hassan JUMAA, Blaubeuren (DE)

(21) Appl. No.: 17/641,605

(22) PCT Filed: Sep. 10, 2019
(86) PCT No.: PCT/EP2019/074120

§ 371 Date: Mar. 9, 2022

(51) Int. Cl. C12Q 1/6886

(52) U.S. Cl. CPC C12Q 1/6886; C12Q 2600/118; C12Q 2600/158

(57) ABSTRACT

The present invention relates to the field of detection and prophylaxis of a tendency to form the symptoms of a CLL, which is based on a mutation of a specific gene used to form the B-cell receptor (BCR). In order to detect said tendency, an allele of the IGVL3-21 gene, the allele IGVL3-21*01, is detected by sequencing or PCR or a different suitable method.

The instant application contains a Sequence Listing, which has been submitted electronically in ASCII format and is hereby incorporated by reference in its entirety. Said ASCII copy, created on Jul. 5, 2022, is named eolf-seqlz_5Jul2022 and is 1,710 bytes in size.

In general, malignant illnesses of the hematopoietic system or the lymphatic system are referred to as malignant B-cell neoplasms. They comprise symptoms such as leukemia, for example, and are part of cancers in the broader sense. Leukemias are characterized by greatly increased formation of non-functional precursor cells of white blood cells, which are also called leukemia cells. These cells spread in the bone marrow, displace normal hematopoiesis there, and generally also accumulate in peripheral blood in a greatly increased manner. They can infiltrate the liver, spleen, lymph nodes, and other organs, and thereby impair their function. Disruption of hematopoiesis leads to a reduction in normal blood components, and thereby anemia can occur due to a deficiency of red blood cells that transport oxygen, as can a deficiency of hemostatic platelets, and a deficiency of mature functional white blood cells.

Depending on the course of the disease, a distinction is made between acute and chronic leukemia. Acute leukemias are life-threatening illnesses that lead to death in a few weeks to months if untreated. Chronic leukemias, in the other hand, generally proceed over several years and are frequently low in symptoms in the initial stage.

The most important forms of leukemia are:
acute myeloid leukemia (AML),
chronic myeloid leukemia (CKL),
acute lymphatic leukemia (ALL),
chronic lymphatic leukemia (CLL).

A familial predisposition to become ill from CLL is known, but it is so rare that there are no genetic correlations for risk factors (Cerhan JR, Slager SL: Familial predisposition and risk factors for lymphoma; Blood 125:2265-2273, 2015; DOI:10.1182/blood-2015-04-537498).

Therefore there is no genetic marker for an illness risk for CLL in the state of the art, and this is true although a hereditary component of the illness is known.

It is the task of the invention to make a method available for determining a hereditary risk of becoming ill with CLL.

The solution for this task lies in the recognition that a somatic mutation that leads to CLL leads to autonomous activation of the BCR and thereby to illness only in the case of one allele of the IGVL3-21 genes, but not in the case of the other two known alleles. Therefore detection of this allele makes it possible to determine an increased risk of becoming ill with CLL.

In more recent investigations, however, it was shown that a special sequence in the light chain of the B-cell receptor (BCR) occurs particularly frequently in what are called autonomously active CLL forms ((IGVL3-21) Stamatopoulos, Basile, et al., “The light chain IGVL3-21 defines a new poor prognostic subgroup in Chronic Lymphocytic Leukemia: results of a multicenter study” Clinical Cancer Research (2018): clincanres-0133). In this regard, a mutation (R110) is present in this light chain, which leads to permanent activation of the BCR (Minici, Claudia, et al., “Distinct homotypic B-cell receptor interactions shape the outcome of chronic lymphocytic leukemia,” Nature Communications 8 (2017): 15746). This mutation is not found outside the B-cell with the expected frequency, neither in healthy test subjects nor in patients. In practice, the mutation is found almost not at all. In patients with CLL, however, it occurs with an unexpected frequency of about 10-15% in the CLL cells, but not in other body cells or non-malignant B-cells. The mutation (R110) permits dimerization of the BCR, which leads to autonomous activation of the receptor (i.e. a ligand that dimerizes these receptors by means of cross-linking is no longer necessary). There are three known alleles of IDVL3-21, which are named 01, 02, and 03. Surprisingly, the allele 01 is over-represented in CLL patients.

The DNA sequence of the alleles is known to a person skilled in the art and can be found, for example, at IMGT (www.imgt.org) under the following Accession Number:
IGVL3-21*01: X71966,
UGVL3-21*02: D97007,
IGVL3-21*03: M94115.

This allele 01 codes for the following amino acid sequence:

SEQ ID 01:
SYVLTQPPSVSVAPGKTARITCGGNNIGSKSV
HWYQQKPGQAPVLVIYYDSDRPSGIPERFSGS
NSGNTATLTISRVEAGDEADYYCQVWDSSSDH

Here it was shown that only the mutated BCR is autonomously active and that the receptor requires the mutation R110 for self-activation. However, this mutation leads to autonomous activation only in the case of the allele 01. This mutation leads to the result that the residues K16 and R110 on one light chain interact with the residues D50 and D52 on the adjacent light chain of a second BCR. In this regard, the residues D50 and D52 must be present as Y49D50S51D52. If the mutation R110 is converted to the wild-type variant G110 by means of genetic manipulation, autonomous activation no longer takes place (Minici et al. 2017). Therefore allele VGL3-21*01 represents a risk factor for CLL illness. The increased (somatic) mutation frequency is explained by the genetic rearrangement that occurs in the formation and maturation of the BCR. Therefore a person who has the risk allele has an increased risk of becoming ill with CLL. In other words, in the case of a person who has the allele 01 of VL3-21, regular precautionary examination can clearly reduce the risk of disease, in particular since antibodies against this mutated epitope of BCR are available. Such a precautionary examination can take place, for example using an antibody that binds to the R110 mutation of the BCR. Such an antibody and its use are described in EP18162666.4. Such tests can be carried out inexpensively by means of flow cytometry, using small amounts of blood. A further possibility is detecting the mutation in DNA or mRNA that was obtained from blood or from cell fractions of blood, by means of Real-Time PCR.

Detection of the genomic presence of VL3-21 can take place directly from DNA, by way of sequencing (for example whole genome sequencing, PCR or other methods). An analysis as to whether VL2-21-01 is present in homozygous or heterozygous form is not necessary in this regard, but can take place for checking purposes (by means of the detection of the other VL3-21 alleles). There are companies that offer genome analysis services (for example 23andme). A service offered by such a company would also be included in the invention described here. If the alleles are supposed to be detected by means of PCR, it is possible to use a probe that detects the risk allele for Real-Time PCR analyses and/or to use multiple probes that detect the other alleles. The alleles *02 and *03 can be detected using such a probe. Therefore positive detection of allele *01 and negative detection of the alleles *02 and *03 is detection of the homozygous risk allele. Positive detection of allele *01 and of a further allele is detection of the heterozygous case. Suitable probes and their use for Real-Time PCR and other methods that use DNA probes are known to a person skilled in the art (Beacons, Scorpion, FRET probes, Light-Cycler probes, etc. https://de.wikipedia.org/wiki/Real_Time_Quantitative_PCR). Further molecular biology methods for detection of the alleles are familiar to a person skilled in the art and comprise, for example, allele-specific PCR, in-situ hybridization, NASBA.

If the risk allele VL3-21*03 was found in test subjects, it is appropriate to take measures of primary prevention. Such measures serve to prevent a disease outbreak. This includes, for example, avoiding unnecessary exposure to radiation (ionizing radiation) and following a balanced diet.

Measures of secondary prevention, which consist of screening and preventive examinations, can be, for example, regular blood tests to determine the lymphocyte count or also analyses based on antibodies, to find the mutated cells.

The present invention will be explained in greater detail below, using examples.

### Example 1

Real-Time PCR for Detecting IGLC3-21*01

A Real-Time PCR test that detects the genetic differences of the alleles is sufficient for detecting the alleles of IGVL3-21. For this purpose, a probe that comprises the regions 129-157 of the IGLV3-21 gene is suitable:

IGLV3-21*01 probe:
(SEQ ID 02)
TGTGCTGGTCATCTATTATGATAGCGACC
IGLV3-21* 02/03 probe:
(SEQ ID 03)
TGTGCTGGTCGTCTATGATGATAGCGACC
IGLV3-21 FW47:
(SEQ ID 04)
AGACGGCCAGGATTACCT
IGLV3-21 RW198:
(SEQ ID 05)
AGAGTTGGAGCCAGAGAATC

In this regard, the probes are modified with a reporter dye at their 5′ end and with a quencher at the 3′ end. In this regard, the IGVL2021*01 probe is marked with the dye FAM, and the IGLV3-21*02/03 probe is marked with the dye TAMRA. The quenchers were selected to match the dyes (BHQ-1 for FAM and BHQ-2 for TAMRA)—These modified DNA probes, with the dyes/quenchers, can be purchased from biomers.net in Ulm.

For detection, extracted genomic DNA (for example from blood, saliva or other tissues) is analyzed by means of the primers and probes.

The precise method of procedure is known to a person skilled in the art, but for the sake of completeness, an embodiment will be stated.
a) Standard protocol:
H₂O bidist. (ad 25 μl)
20% glycerin: 8%
10× TaqMan Buffer A: 1×
25 mM MgCl12 solution: 5.0 mM
dATP: 200 μM
dCTP: 200 μM
dGTP: 200 μM
dUTP: 200 μM
Primer 1: 200 nM
Primer 2: 200 nM
Probe allele 1: 100 nM
Probe allele 2/3: 100 nM
AmpliTaq Gold (5 U/mL): 1.25 U
AmpErase UNG: 0.25 U
DNA: approximately 10,000 starting copies
PCT programs
UNG digestion: 50° C. for 2 min
AmpliTaq Gold activation: 95° C. for 10 minutes
40 cycles: 95° C., 15 sec
   60° C., 1 min

The evaluation takes place using the CT values of the individual color channels, with CT values below 32 (<32) being interpreted as a positive signal for the allele. If fewer copies of genomic DNA are used, the limits of the CT values can be lower.

### Example 2

FACS Test for the Autonomously Active IGLV3-21*01 R110

A small amount of peripheral blood was taken from a patient. For the analysis, 100 μL of blood were transferred to a reaction vessel and 2 mL of PBS-BSA buffer solution were filled in. Subsequently the sample was centrifuged in an Eppendorf centrifuge 5804 at 1500 rpm for a time of five minutes. The top fraction was discarded, and the sediment was mixed thoroughly. Subsequently the antibody was added. Dyeing was performed against the following surface parameters: 1) CD19-FITC, 2) CD5-PE, and 3) the antibody specific to the CLL subset-2 (APC), before the batches were incubated at room temperature, in the dark, for 15 minutes. Afterward, lysis was initiated, and the erythrocytes were lysed. As already described above, washing with PBS-BSA buffer solution took place twice, and the cells were incorporated into 500 μL 0.1% PBS-BSA buffer solution and resuspended. Until being measured in the flow cytometer, the cells were kept in the dark at 2-8° C.

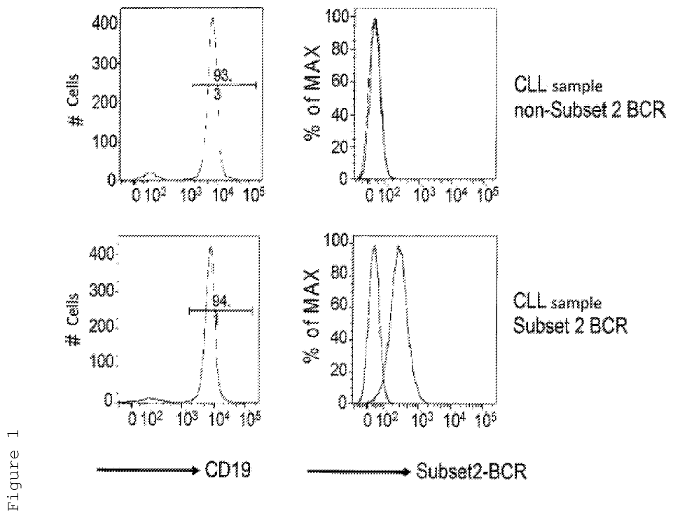

The analysis using FACS took place on a BDCalibur. Adjustment of the individual laser and detection parameters took place in accordance with manufacturer instructions and is sufficiently known to a person skilled in the art. The raw data of the analysis were subsequently evaluated by means of FlowJo analysis software. First the lymphocyte population in the FSC/SSC blot was selected and marked. For this selection the focus was then on the CD19-positive B-cells, and the analysis looked for binding of the antibody specific for subset-2. Such an analysis is shown as an example in FIG. 1, using the example of use of the antibody specific for subset-2. In a first step, the CD19 positive B-cells were selected for further analysis (left panel). These were then checked for binding of the specific antibody.

1. A method for diagnosing a predisposition for leukemia in humans, which comprises determining, in a DNA sample of a human, whether a DNA sequence is present that codes for a polypeptide according to SEQ. ID. No. 1, wherein the presence of this DNA sequence shows a predisposition for leukemia.

2. A method for diagnosing a predisposition for leukemia in humans, which comprises determining, in a DNA sample of a human, whether a DNA sequence is present that codes for a polypeptide that has the following amino acids in the following positions: Y49, D50, S51, and D52, wherein the presence of this DNA sequence shows a predisposition for leukemia.

3. A method for diagnosing a predisposition for leukemia in humans, which comprises determining, in a DNA sample of a human, whether a DNA sequence is present that codes for a polypeptide that has the following amino acids in the following positions: K16, Y49, D50, S51, and D52, wherein the presence of this DNA sequence shows a predisposition for leukemia.

4. The method according to claim 1, wherein the DNA sequence is determined by means of sequencing.

5. The method according to claim 1, wherein the DNA sequence is determined by means of PCR.

6. The method according to claim 5, wherein the DNA sequence is determined by means of a reporter probe.

7. The method according to claim 6, wherein it is determined whether the allele IGVL3-21*01 is present in homozygous or heterozygous form.

8. The method according to claim 1, wherein the DNA sequence is determined by means of hybridization.

9. The method according to claim 2, wherein the DNA sequence is determined by means of sequencing.

10. The method according to claim 3, wherein the DNA sequence is determined by means of sequencing.

11. The method according to claim 2, wherein the DNA sequence is determined by means of PCR.

12. The method according to claim 3, wherein the DNA sequence is determined by means of PCR.

13. The method according to claim 11, wherein the DNA sequence is determined by means of a reporter probe.

14. The method according to claim 12, wherein the DNA sequence is determined by means of a reporter probe.

15. The method according to claim 13, wherein it is determined whether the allele IGVL3-21*01 is present in homozygous or heterozygous form.

16. The method according to claim 14, wherein it is determined whether the allele IGVL3-21*01 is present in homozygous or heterozygous form.

17. The method according to claim 2, wherein the DNA sequence is determined by means of hybridization.

18. The method according to claim 3, wherein the DNA sequence is determined by means of hybridization.
